(19) United States
(12) Patent Application Publication
(10) Pub. No.: US 2019/0277755 A1
(43) Pub. Date: Sep. 12, 2019

(54) DEVICE AND METHOD FOR TISSUE DIAGNOSIS IN REAL-TIME

(71) Applicant: PIMS- PASSIVE IMAGING MEDICAL SYSTEMS LTD, Emek Hefer Industrial Park (IL)

(72) Inventors: Yaniv COHEN, Jerusalem (IL); Ben Zion DEKEL, Hadera (IL); Arkadi ZILBERMAN, Beer Sheva (IL); Nathan BLAUNSTEIN, Beer Sheva (IL)

(73) Assignee: PIMS- Passive Imaging Medical Systems Ltd, Emek Hefer Industrial Park (IL)

(21) Appl. No.: 16/314,665

(22) PCT Filed: Jul. 5, 2017
(86) PCT No.: PCT/IL2017/050750

§ 371 Date: Jan. 1, 2019

Related U.S. Application Data
(60) Provisional application No. 62/358,173, filed on Jul. 5, 2016.

(51) Int. Cl. G01N 21/3563; G01N 21/552; G01N 21/47; G01N 21/93; G01N 33/483; G01N 1/30; G01J 3/42

(52) U.S. Cl. CPC G01N 21/3563; G01N 21/552; G01N 21/474; G01N 21/93; G01J 2003/421; G01N 1/30; G01J 3/42; G01N 2021/4761; G01N 2021/3572; G01N 33/4833

(57) ABSTRACT

A device for real-time tissue diagnosis of biological tissue having: a means for preparing a tissue sample before a measurement procedure; a means for positioning an ATR element and mirrors so as to perform a system calibration; a means for irradiating a sample with IR radiation using the ATR element and an opto-mechanical assembly; a means for recording the absorption spectrum of a sample being tested; a means for carrying out a Fourier transformation of the absorption spectrum obtained into a FT-IR spectrum; a means for calculating tissue characteristics on the basis of signal processing; a means for comparing the characteristics in a pre-selected wavenumber range with the reference spectra prepared and stored in a database. Also, a method for real-time tissue diagnosis of biological tissue having solely the following steps: setting operating parameters: scanning ambient background air to obtain a background spectrum; placing a tissue under test in tight contact with an ATR; drying the tissue so as to at least reduce moisture content of the tissue sample; automatically adjusting at least one system mirror thereby performing a system calibration; and obtaining a spectrum of the tissue sample.

### FIELD AND BACKGROUND OF THE INVENTION

The present invention relates generally to infrared (IR) spectroscopy devices, systems and methods for in-vitro/ex-vivo analyzing and/or identifying biological tissue/cells and fluids. More specifically, the present invention is a device for tissue diagnosis in real-time and method for use with the device.

Infrared (IR) spectroscopy is a technique routinely used by the biochemists, material scientists etc. as a standard analyzing method. The observed spectroscopic signals are due to the absorption of infrared radiation that is specific to functional groups of the molecule. These absorption frequencies are associated with the vibrational motions of the nuclei of a functional group and show distinct changes when the chemical environment of the functional group is modified.

IR spectroscopy essentially provides a molecular fingerprint and IR spectra contain a wealth of information on the molecule. In particular they are used for the identification and quantification of molecular species, the interactions between neighboring molecules, their overall shape, etc. IR spectra can be used as a sensitive marker of structural changes of cells and of reorganization occurring in cells.

Organic applications of IR spectroscopy are almost entirely concerned with frequencies in the range of 4000 cm⁻¹ to 400 cm⁻¹ (2.5 um to 25 um), which is known as mid-infrared (MIR) region of the spectrum. The range of frequencies lower than 400 cm⁻¹ is called far-infrared (FIR) and those greater than 4000 cm⁻¹ are called near-infrared (NIR).

In MIR range occur most of the fundamental molecular vibrations and many of the first overtones and combinations. The bands in the mid-infrared tend to be sharp and have very high absorptivities, with both characteristics being desirable. Because the bands are sharp, most small molecules have distinctive spectral “fingerprints” that can be readily identified in mixtures. Also, because individual peaks can often be associated with individual functional groups, it is possible to see changes in the spectrum of an individual “objects” due to a specific reaction.

Most biomolecules give rise to IR absorption bands between 1800 cm⁻¹ and 700 cm⁻¹, which is known as the “fingerprint region” or primary absorption region.

The present invention relates to methods employing Evanescent Wave Fourier Transform Infrared (EW-FTIR) spectroscopy using optical elements and sensors operated in the attenuated total reflection (hereinafter referred to as “ATR”) regime in the MIR region of the spectrum.

FTIR can be used to detect vibration in chemical bonds and, as such, it is used to sense the biochemical composition of tissues. Although not capable of detecting specific molecules because many bond vibrations are shared among biomolecules, FTIR can be used to quantify classes of molecules (i.e. glycogen, protein, fat or nucleic acid etc.). FTIR has largely been performed on excised tissues and used to demonstrate that the overall biomolecular composition of diseased tissues is altered in a predictable manner relative to that of adjacent normal tissue.

Unlike conventional methods, FTIR-ATR spectroscopy in the MIR region of the spectrum probes tissue biochemistry at a molecular level and the observed MIR spectra exhibit superimposed or composite vibrational bands. Large biomolecules are represented in FTIR-spectra by groups of characteristic IR-bands from which valuable information can be gained regarding the structure of the molecule and its interactions depending on position, form (shape), and intensity.

Therefore, the present combined apparatus can be applied to many fields: (i) medical diagnostics of cancer and other disease states ex-vivo/in-vitro, (ii) monitoring of biochemical processes, (iii) surface diagnostics of numerous materials, (iv) characterization of the quality of food, pharmacological products and cosmetics, liquids, etc.

The present invention provides a method to detect functional molecular groups to elucidate complex structure within tissue, to characterize, distinguish and diagnose healthy, tumorous, precancerous, and cancerous tissue at an early stage of development.

Typically, cancer occurs when a normal cell undergoes a change which causes the cell to multiply at a metabolic rate for exceeding that of its neighboring cells. Continued multiplication of the cancerous cell frequently results in the creation of a mass of cells called a tumor. Cancerous tumors are harmful because they grow at the expense of normal neighboring cells, ultimately destroying them. In addition, cancerous cells are often capable of traveling throughout the body via the lymphatic and circulatory systems and of creating new tumors where they arrive. It should be noted that in addition to tumors which are cancerous (also referred to as malignant tumors) there are tumors which are non-cancerous. Non-cancerous tumors are commonly referred to as benign tumors. It is useful to be able to determine whether a tumor is cancerous of benign.

The present invention relates to the IR spectroscopic method for determining if a tissue is a malignant tumor tissue, a benign tumor tissue, or a normal or benign tissue.

The invention also includes nontoxic, ex-vivo, and fast (real-time) characterization of normal and abnormal tissue from breast, stomach, lung, prostate, kidney and other body parts during surgery, allowing an alternative first step of spectral histopathological examination and disease state characterization.

The method of the invention may be used for ex-vivo/in-vitro analysis, e.g., examination, determination, classification, identification, typification, differentiation, monitoring, quantification, diagnosis and/or as a status check.

### SUMMARY OF THE INVENTION

The present invention is device for tissue diagnosis in real-time and method for use with the device.

The aim of present invention is to develop a dedicated combined apparatus suitable for biological tissue characterization via FTIR spectroscopic measurement during clinical practice.

In particular, the present invention provides apparatus suitable for cancer diagnosis. Generally, the method of the invention is based on FTIR-ATR spectroscopy.

According to the teachings of the present invention, the invention relates to combined device and method for the in-vitro analysis of tissue and biological cells which may be carried out in a simple and, preferably, automated manner. The device and method produces result rapidly (up to minutes) and permits the determination/detecting of structural changes between a biological specimen and a reference sample.

In accordance with the teachings of the present invention the tissue of animals and humans are applied to unclad optical element (crystal, etc.) working in ATR regime. A beam of infrared radiation (preferably mid-IR) is passed through a low loss optical element and interacts with the tissue via the ATR effect. In this process, the absorbing tissue is placed in direct contact with the optical element.

According to the teachings of the present invention, the apparatus of the invention is a device consisting FT-IR spectrometer with a set of optical and opto-mechanical elements.

The novel combined apparatus (FTIR spectrometer with opto-mechanical elements and Software) adopts an integrative design in appearance, and is configured as a bench top device as illustrated is FIG. 5.

According to the teachings of the present invention there is provided, a device for real-time tissue diagnosis of biological tissue, the device comprising: a means for preparing a tissue sample before a measurement procedure; a means for positioning an ATR element and mirrors so as to perform a system calibration; a means for irradiating a sample with IR radiation using the ATR element and an opto-mechanical assembly; a means for recording the absorption spectrum of a sample being tested; a means for carrying out a Fourier transformation of the absorption spectrum obtained into a FT-IR spectrum; a means for calculating tissue characteristics on the basis of signal processing; a means for comparing the characteristics from the previous step in a pre-selected wavenumber range with the reference spectra prepared and stored in a database.

According to the further teachings of the present invention there is also provided that the means for preparing a tissue sample before a measurement procedure includes a tissue drying arrangement configured at least reduce moisture content of the tissue sample.

According to the further teachings of the present invention there is also provided that the means for preparing a tissue sample before a measurement procedure includes a pressure device for ensuring good contact between the tissue sample and the ATR element.

According to the further teachings of the present invention there is also provided that all mechanical components are deployed in a single portable benchtop housing.

There is also provided according to the further teachings of the present invention a device for real-time tissue diagnosis of biological tissue, the device comprising at least an ATR element, mirrors, and a calibrating assembly with an automatic mirror position adjuster configured to adjust a position of at least one of the mirrors so as to perform a system calibration.

There is also provided according to the further teachings of the present invention a method for real-time tissue diagnosis of biological tissue, the method solely comprising the following steps: setting operating parameters; scanning ambient background air to obtain a background spectrum; placing a tissue under test in tight contact with an ATR; drying the tissue so as to at least reduce moisture content of the tissue sample; automatically adjusting at least one system mirror thereby performing a system calibration; and obtaining a spectrum of the tissue sample.

According to the further teachings of the present invention there is also provided that the setting operating parameters includes setting at least one of scanning rate; the number of scans; the resolution of the apparatus; and spectral range.

According to the further teachings of the present invention there is also provided that the automatically adjusting at least one system mirror thereby performing a system calibration is a precise adjusting of a mirror position based on one of (a) a maximal signal for RAW data; (b) absorbance values; and (c) maximal SNR.

### BRIEF DESCRIPTION OF THE DRAWINGS

The invention is herein described, by way of example only, with reference to the accompanying drawings, wherein:

FIG. 1 is an illustration showing total internal reflection of IR waves;

FIG. 2 is an illustration showing evanescent wave action at the interface between two media, under total internal reflection conditions;

FIG. 3 is a graph illustrating the depth of penetration, dp, of the evanescent wave into the sample for a ZnSe-sample interface at different incidence angles (in degrees);

FIG. 4 is a graph illustrating the ATR spectrum of biological tissue;

FIGS. 5A and 5B are photographs of the benchtop embodiment of the FTIR-ATR device of the present invention showing the access lid closed and open, respectively;

FIG. 6 is a block diagram of the FTIR-ATR device of the present invention;

FIG. 7 is a photograph of the FTIR-ATR device of the present invention deployed in the benchtop case of FIGS. 5A and 5B;

FIG. 8 is a block diagram of the ATR device assembly of the present invention;

FIG. 9 is an illustrative graph comparing normal tissue and cancer tissue;

FIG. 10 is an illustrative graph for Principal Component Analysis training;

FIG. 11 is an illustrative graph of 3D Support Vector Machine used for both training and classification; and

FIG. 12 is a flow chart of the method of the present invention.

### DESCRIPTION OF THE PREFERRED EMBODIMENTS

The present invention is device for tissue diagnosis in real-time and method for use with the device.

The principles and operation of the device and method according to the present invention may be better understood with reference to the drawings and the accompanying description.

By way of introduction, FT-IR is a method of obtaining infrared spectra by first collecting an interferogram of a sample signal using an interferometer, and then performing a Fourier Transform (FT) on the interferogram to obtain the spectrum.

The detection scheme is based on a Michelson interferometer, where a moving mirror varies the length of one optical path relative to the other, and creates an interferogram that is mathematically converted to an absorbance spectrum by a Fourier transform.

As the optical path difference (OPD) in the interferometer grows, different wavelengths produce peak readings at different positions.

FTIR spectroscopy is based on the interaction between the radiation and the sample, which absorbs the IR wavelengths causing transitions between vibrational energetic levels; therefore, vibrational modes of different chemical bonds can be detected and allow to identify different molecules.

EW-FTIR spectroscopy is based on the phenomenon of attenuated total reflection (ATR).

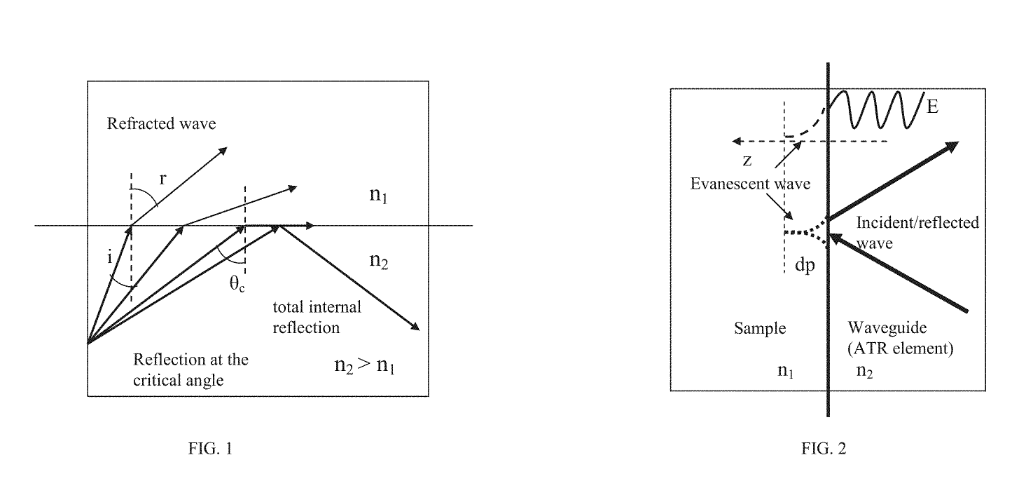

ATR spectroscopy utilizes total internal reflection phenomenon. An internal reflection occurs when a beam of radiation enters from a more dense medium with a higher refractive index, n2, into a less-dense medium with a lower refractive index, n1. When the angle of incidence is greater than the critical angle θc (θc=sin−1n1/n2) or when the angle of refraction exceeding 90°, all incident radiations are completely reflected at the interface, results in total internal reflection, as illustrated in FIG. 1 where “i” is the angle of incidence and “r” is the angle of refraction.

In ATR spectroscopy a crystal with a high refractive index and IR transmitting properties is used as an internal reflection element (ATR crystal). The ATR element is placed in contact with the sample. See FIG. 2 which illustrates the evanescent wave formed at the internal reflection element-sample interface. The beam of radiation propagating in ATR undergoes total internal reflection at the interface ATR-sample, provided the angle of incidence at the interface exceeds the critical angle θc. Total internal reflection of the light at the interface between two media of different refractive index creates an “evanescent wave” that penetrates into the medium of lower refractive index. “Evanescent” means “tending to vanish”, which is appropriate because the intensity of evanescent waves decays exponentially with distance from the interface at which they are formed. This distance is typically in the 1-10 um range.

The depth of penetration of the evanescent wave is a function of the refractive index of the optical element material, refractive index of the sample material, angle of incidence of the radiation wavefront, and wavelength of the radiation. In regions of the spectrum where the sample absorbs energy, the evanescent wave is attenuated and the attenuated energy is passed back to the optical element. The radiation then exits the optical element and impinges a detector through optical waveguide/fiber. The detector records the attenuated radiation, which can then be transformed to generate a spectrum, e.g., absorption spectra.

The evanescent field is a non-transverse wave along the optical surface, whose intensity decreases with increasing distance into the medium, normal to its surface, therefore, the field exists only the vicinity of the surface. The exponential decay evanescent wave can be expressed by:

E_{EW}=E₀ exp(−z/dp),

where z is the distance normal to the optical interface, dp is the penetration depth, and E0 is the intensity at z=0.

The depth of penetration, dp, of the evanescent wave into the sample is defined as the distance from the ATR-sample boundary where the intensity of the evanescent wave decays to 1/e (37% of its original value), it is given by:

dp=(n_{ATR}² sin²θ−nₛ²)^{-1/2}/k,k=2π/λ

where λ is wavelength of the radiation, θ is the angle of incidence of the wavefront (light beam), nATR and nS are the refractive indices of ATR element and sample respectively.

The sensing mechanism is based on the absorption of the evanescent electric field, which propagates outside the surface of the waveguide (crystal, fiber) and interacts with any absorbing species at the waveguide-sample interface.

The sample under test is placed in tight contact with the ATR along the IR radiation pathway, between the source and the detector side.

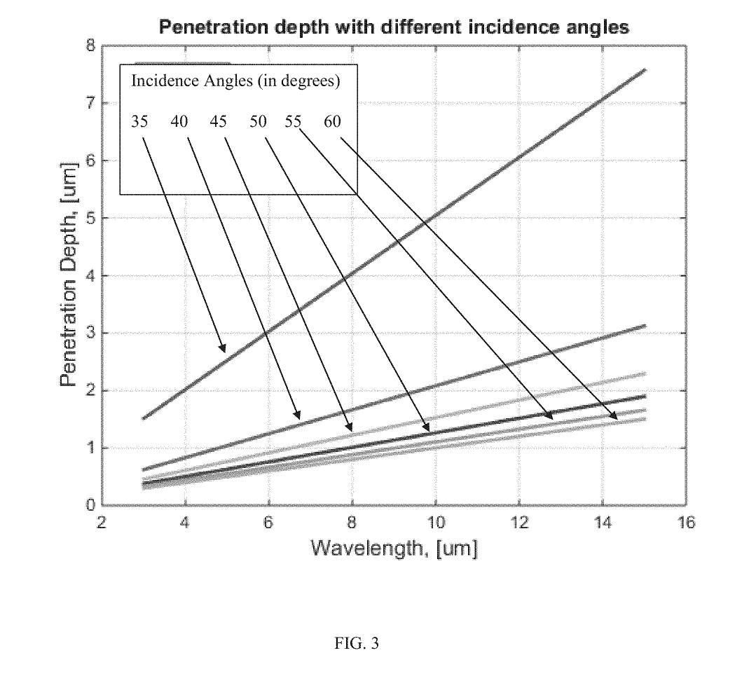

FIG. 3 illustrates the depth of penetration, dp, of the evanescent wave into the sample for a ZnSe-sample interface and different incidence angles (in degrees) where nATR=2.4, nS=1.34 and θc=34o.

It is therefore an object of the invention to provide a convenient means of positioning the ATR element and mirrors.

When the optical element (ATR crystal/fiber) is in contact with a tissue sample, the evanescent wave is either partially or totally absorbed at specific absorption lines as determined by the biochemical composition of the tissue. The total transmission through the waveguide (ATR element) and the tissue sample will decrease at the absorption lines that correspond to molecular bonds in particular classes of bio-molecules.

Consequently, only tissue that is in good optical contact with the ATR element can be sampled. Despite relatively high concentrations in the bulk tissue, components with poor optical contact can be difficult to measure in the ATR spectrum.

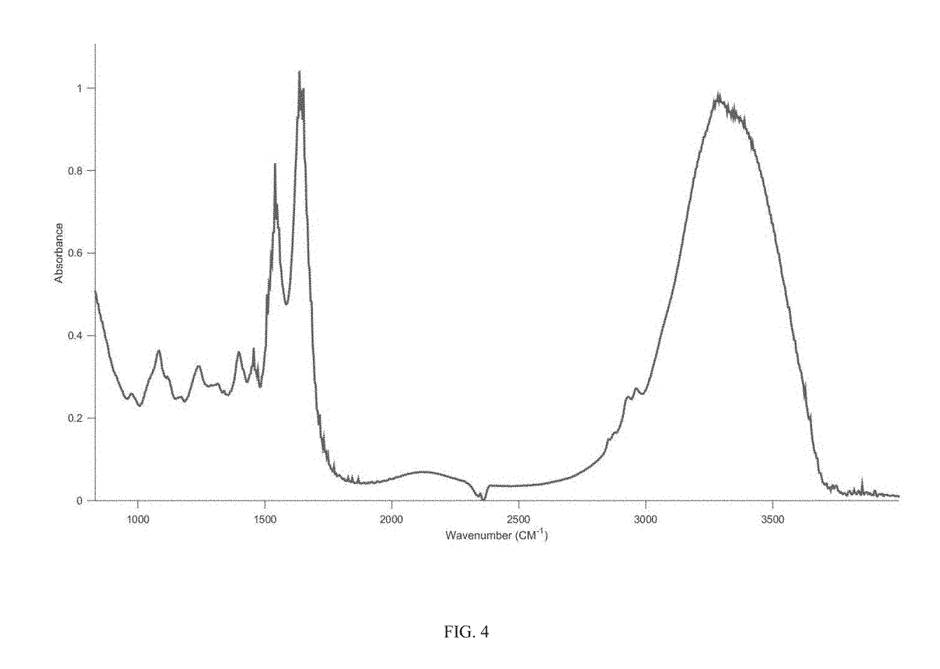

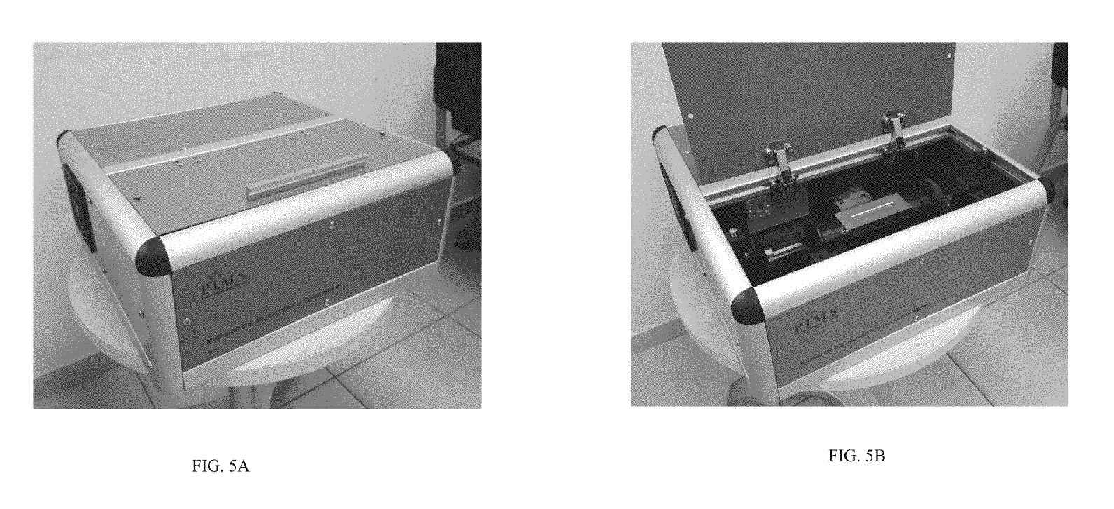

An ATR spectrum can be obtained by measuring the interaction of the evanescent wave with the sample. If an absorbing material is placed in contact with ATR crystal, the evanescent wave will be absorbed by the sample and its intensity is reduced (attenuated) in regions of the IR spectrum where the sample absorbs, thus, less intensity can be reflected (attenuated total reflection). The resultant attenuated radiation as a function of wavelength produces an ATR spectrum which is similar to the conventional absorption spectrum except for the band intensities at longer wavelengths (FIG. 4). This difference is due to the dependency of the penetration depth (dp) on wavelength: at longer wavelength, the evanescent wave penetrates deeper into the sample (see FIG. 3), thus, the absorption bands at longer wavelengths are relatively more intense than those shorter wavelengths. This results in greater absorption on the longer wavelength side of an absorption band, contributing to band distortion and band broadening.

FIG. 4 illustrates the ATR spectrum of biological tissue. Here below are typical IR absorbance positions: the bands around ˜1640 cm⁻¹ and ˜1550 cm⁻¹—protein absorption region (Amide I and Amide II); the bands around ˜1480 cm⁻¹ and ˜1400 cm⁻¹—lipids and protein absorption region (CH3); the bands between 1000-1300 cm⁻¹, PO2 symmetrical and asymmetrical stretching vibrations, indicate changes for phospholipids and nucleic acids; phospholipids and Amide III at ˜1240 cm⁻¹; CO stretching at ˜1160 cm⁻¹; the bands between 2800-3100 cm⁻¹ (the stretching vibrations of lipid hydrocarbons); the peaks around ˜2850 cm⁻¹ and ˜2923 cm⁻¹ indicate enhancement in lipid contents; the peak around 2350 cm⁻¹ is the carbon dioxide absorption (CO2); and the peak around ˜3150-3600 cm⁻¹ strong water absorption.

FT-IR analysis allows recognition of the presents of functional groups in molecules, both organic (e.g. CH2, C═O, etc. groups), and inorganic (carbonates, sulphates, etc.). The characterization is performed through comparison with spectral data contained in databases (digital spectral databases) and assigning each peak to the vibration of a specific functional group.

The mode of operation of the present invention is as follows:

The present invention provides an apparatus and method for ex-vivo/in-vitro analysis of biological cells and/or tissue characteristics that includes:

a means for preparing sample before the measurement;

a means for positioning the ATR element and mirrors so as to provide automatic system calibration;

a means for irradiating a sample with IR radiation using an ATR element and opto-mechanical assembling;

a means for recording the absorption spectrum;

a means for carrying out a Fourier transformation of the absorption spectrum obtained into a FT-IR spectrum;

a means for calculating the tissue characteristics on the basis of sophisticated signal processing (spectral analysis and contrast, chemometric, etc.); and

a means for comparing the characteristics from the step (f) in a pre-selected wavenumber range with the reference spectra prepared and stored in the database of the apparatus, or a database available to the apparatus.

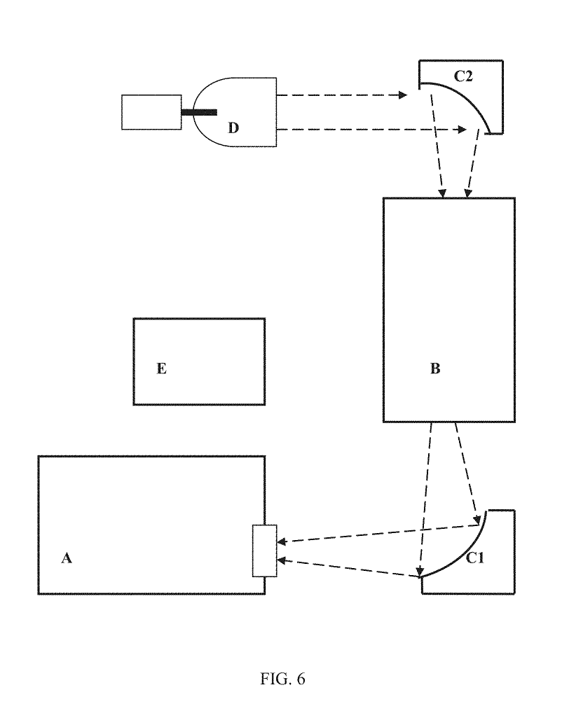

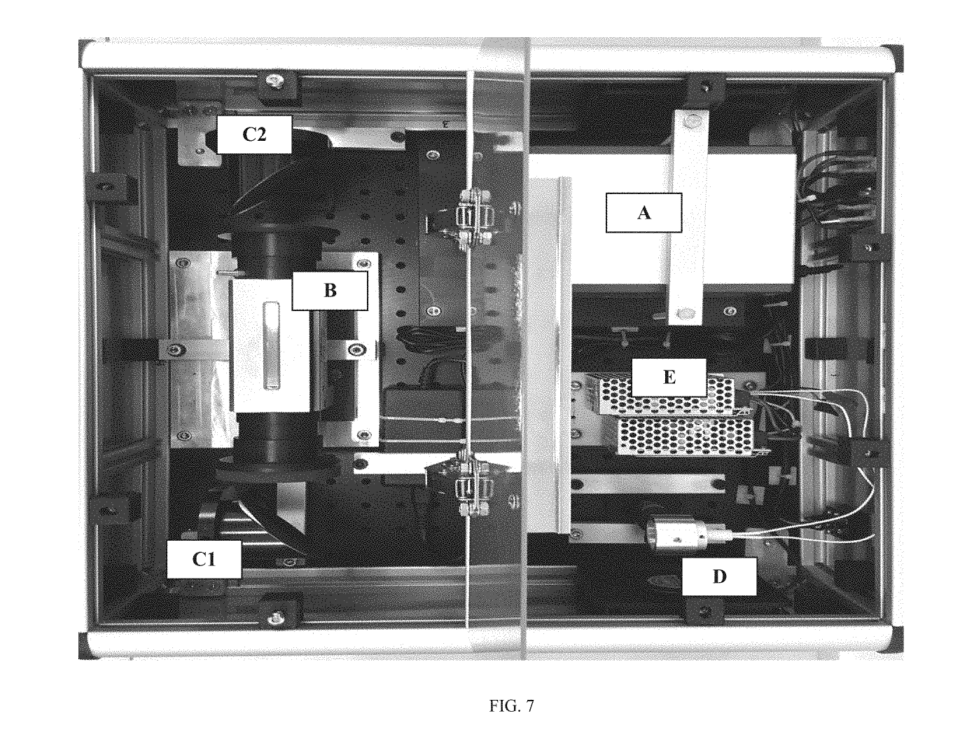

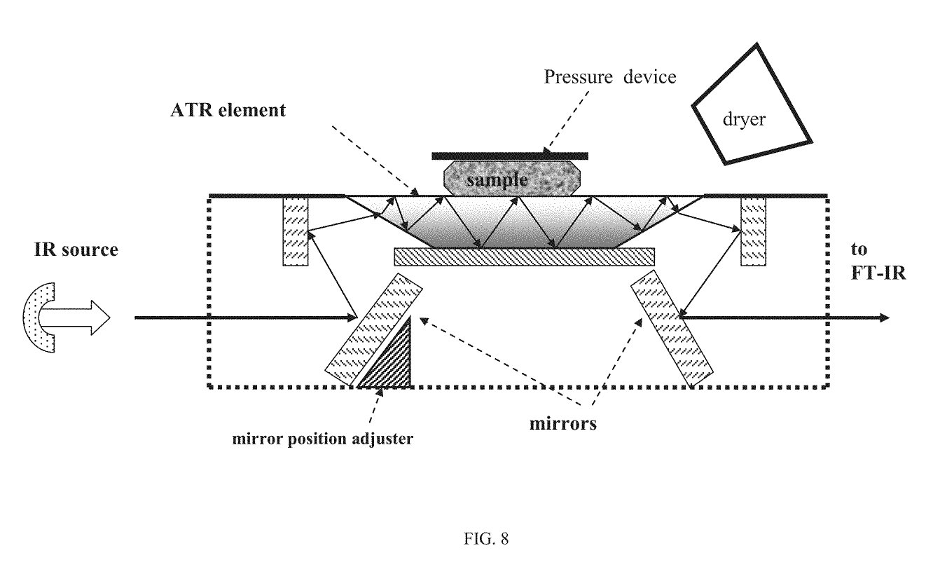

Referring now to the drawings, the method of the invention is described in connection with the block drawing of the device of the present as shown in FIG. 6 and the photograph of a prototype of a preferred embodiment of the present invention shown in FIG. 7.

The optical system consists of the removable optical element (B) configured for input and output of the infrared radiation, mirrors C1 to focus an infrared beam into the optical element and C2 to direct light from the optical element onto the input of the FTIR spectrometer (A) which includes a cooled detector (e.g. MCT detector etc.). The optical scheme of the invention is specifically designed and applicable with commercial FTIR spectrometers.

As illustrated, the opto-mechanical elements of the dedicated apparatus include a mid-IR ATR element and ATR device assembly (B), mirrors with holder components (C1 & C2), and an infrared source part (D) as explained below. The electrical part of the apparatus also includes a power supply (E).

Infrared reflectors (parabolic mirrors, C1 & C2) are placed at both ends of optical construction with ATR element (B) to provide the transmitted light from IR source (D) through the ATR element to the detecting part (FT-IR spectrometer, A). The ATR element is a separate component fastened to an optical construction (see FIG. 7 and FIG. 8).

The ATR element (crystal) is a flat rod of high refractive index material which acts as a waveguide for the infrared sampling beam. This waveguide can have geometry suitable for contacting the surface of tissue.

The ATR can be constructed from the optical fiber by removing the cladding material.

Sampling is provided by placing the tissue of interest in optical contact with the ATR element. Radiation is transmitted and collected in a radial direction from the element as illustrated by the block diagram of FIG. 8. It should be noted that FIG. 8 also illustrates the features of a pressure device for ensuring good contact between the sample and the ATR element, and a dryer for reducing the moister content of the sample before testing.

The evanescent wave which extends outside of ATR element surface is absorbed by the sample in proportion to its absorption properties (quantified by absorption coefficient). As was mentioned above, the penetration depth of the evanescent wave into the sample depends on the wavelength of the IR radiation and the refractive indices of the ATR element and the sample; e.g., for ZnSe-water interface, this depth is roughly 4-10 micron from 1800 to 700 cm⁻¹.

FIG. 8 illustrates an ATR device assembly that includes a removable ATR element, mirrors, and calibrating assembly with automatic mirror position adjuster.

In accordance with a feature of the invention, the ATR element is used to interface with the sample for measuring infrared spectral response of the sample.

In the illustrated embodiment (FIG. 8), the ATR element is a flat ZnSe crystal (45o ends). The interface between the sample and the ATR during an infrared reflection measurement is shown in the FIG. 8. The ATR includes an ATR crystal which has a higher index of refraction for infrared light than the sample. Typically, the ATR crystal is made from ZnSe.

To measure infrared spectral response, the sample is brought into contact with the ATR crystal surface. An infrared beam is then directed to the sample through the ATR crystal at a pre-selected angle (defined by the automatic mirror position adjuster in FIG. 8) which is greater than a critical angle so that total internal reflection occurs at the interface of the crystal and the sample.

The calibration process is performed before the measurement cycle. The calibration includes: a) precise adjusting of a mirror position up to maximal signal for RAW data (instrument readings) or for absorbance values (absolute or relative, FIG. 4) or for maximal SNR (; b) fixation of the mirror position.

For the SNR estimation, the calibration signal uses the noise signal measured without the IR source.

For absorption estimation, the air background with IR source is scanned to obtain a background and source spectrum without a sample.

The normalized RAW data:

$$R ( λ ) = \frac{I ( λ ) - Dark ( λ )}{REF ( λ ) - Dark ( λ )} ,$$

REF(λ) is the reference signal of IR source without sample;

Dark(λ) is the dark counts or noise signal;

I(λ) is the spectral intensity (e.g., numbers, instrument readings, etc.) measured with the sample placed on ATR.

The SNR is defined as:

$$SNR = \frac{\text{<} I ( λ ) \text{>}}{σ}$$

where <I(λ)> is the mean signal at appropriate/given wavelengths; a is the standard deviation or RMS of noise signal, Dark(λ).

For the SNR calibration the bands around ˜1640 cm⁻¹ and ˜1550 cm⁻¹ (protein absorption region Amide I and Amide II) can be chosen for the mean signal <I(λ)> or absorption estimation.

The electromagnetic (EM) field of the infrared beam extends into the sample for a short distance. The strength of the EM field in the sample decreases exponentially with the distance from the sample-ATR interface. The EM field in the sample is absorbed by the sample at energies where the sample material is absorptive. The reflected IR light from the sample thus carries information of the absorption characteristics of the sample. The reflected IR light passes through the ATR crystal and is collected by the FTIR spectrometer.

For mid-infrared, the penetration depth is typically on the order of 1 to 10 microns. A 400-900 grams weight placed on the tissue sample ensured uniform sample contact with the ATR element.

Biological samples after pressure typically have a thickness on the order of 100 microns and more and are therefore suitable for measurements by FTIR-ATR spectroscopy.

Basic elements of the developed device:

1) ATR Device Assembly & Removable ATR Element

An infrared beam is directed to the sample through the ATR crystal at a pre-selected angle which is greater than a critical angle so that total internal reflection occurs at the interface of the crystal and the sample. The depth of penetration of the evanescent wave into the sample depends on angle of incidence at ATR-sample interface. The incidence angle is defined by the automatic mirror position adjusting for the maximal S/N ratio.

The ATR element may have different shapes (e.g. flat parallelepiped etc.) with an inclined ends. The sizes (height, length, width) can have arbitrarily set according to technical and clinical requirement.

Materials for the ATR may be ZnSe, Ge, ZnS, Si, halide, sulfide or mixtures thereof. The spectral range is between 700 cm⁻¹ and 4000 cm⁻¹.

A crystal material must have a high index of refraction to allow internal reflectance. Materials with a refractive index greater than 2.2 are normally chosen as ATR crystals.

The material which is most commonly used for ATR spectroscopy is ZnSe. ZnSe has a refractive index of 2.4 making it suitable for most organic materials and it has a transmission range from 20,000 to 650 cm⁻¹. This material is insoluble in water but should not be used in acidic or strong alkaline solutions.

In one embodiment, the claimed invention may use the properties of core-only optical fiber to produce ATR element. A core-only silver halide fiber can be used as ATR element. The polycrystalline silver halide (e.g. AgClxBr1-x) fibers are among the most useful ones for applications in the mid-IR. These fibers have a wide transparency range (˜2-20 um wavelength), they are non-toxic, flexible and insoluble in water. Advances in polycrystalline silver halide optical fibers are enabling clinical applications of FTIR for tissue analyses. They operate with low optical losses (0.1-0.5 dB/m in the region of 10 um) and high flexibility (R-bending>10-100 fiber diameters) in the spectral range of 3-20 um.

The length of interaction of the tissue surface with optical element varies from about 1 to 10 mm. The depth of penetration of the IR radiation in tissue is of the order of the wavelength used.

2) Mirrors and Automatic System Calibration Mechanism

The mirrors and the automatic system calibration mechanism include an abaxial parabolic mirrors (e.g., Off-Axis Parabolic Gold Mirrors) and a precise fine-tuning mechanism. There are 2 pieces of abaxial parabolic mirrors. It serves to bring the parallel infrared light from the infrared source into the input end of ATR and from the output end of ATR into the FTIR spectrometer and detector.

The precise fine-tuning mechanism may be configured as a screw position adjustor for adjusting the position of one or both of the parabolic mirrors to focus the lights precisely into the ATR and detector (input of FTIR spectrometer).

The abaxial parabolic mirror converges the parallel light onto the detector. The 3-dimensional tuning mirror holder serves to adjust the infrared light onto the detector thereby maximizing the light energy accepted by the detector so that satisfactory FTIR spectra with high signal-to-noise ratio (S/N) can be obtained.

3) Infrared Source, Such as, but not Limited to, a Scitec Instruments Inc. IR-18 with 1″ Porabolic Reflector, 12 V, 1.5 A, 18 W Electric Power, T=1150° C.

4) Dehydration/Drying and Pressure Device

One of the main difficulties in measuring mid-IR spectra of bio-tissue is the intense water absorption, which dominates and obscures the absorption of other tissue components of interest.

In preferred embodiment of the device, the tissue is blown with air to minimize interference from water.

Another factor which affects the quality of an ATR spectrum is the efficiency of sample contact. Because the evanescent wave decays very rapidly with distance from the surface, it is important to have the sample in intimate contact with the crystal. This is easily achieved with most liquids since they wet the surface of the ATR crystal. For soft tissue and solid materials, it is important to use a pressure device which presses the sample against the crystal. A 400900 grams weight placed on the tissue sample ensured uniform sample contact with the ATR element.

5) FT-IR Spectrometer

FTIR spectrometer includes an interferometer where the detector signals are obtained as an interferogram and converted by means of Fourier transformation into IR spectrum.

Instrumentation and apparatus for performing FTIR and ATR measurements are available from a number of commercial suppliers, including, but not limited to, Nicolet Instruments Corp., Perkin-Elmer, Bruker Instruments, Inc., Bio-Rad Digilab Division, Spectra-Tech, Inc.

The preferred embodiment for the device—the compact FTIR spectrometer from Arcoptics (Switzerland), model FTIR Rocket 2-12 μm. Arcoptics FTIR spectrometer includes an infrared detector and interferometer assembly for providing mid-infrared energy suitable for infrared spectroscopic studies. The main features: Compact (world smallest FTIR), resolution 4 cm⁻¹, Peltier cooled MCT detector D*[cm Hz1/2W−1]>1.5×109, 2.5-12 um spectral range.

Detection according to the inventive method is comprised of the following steps:

1) Operating parameters of the apparatus are set. Said operating parameters include: scanning rate, the number of scans, the resolution of the apparatus and spectral range.

The required resolution of the apparatus is 4 cm⁻¹ or 8 cm⁻¹, and the number of scans may be more than one. Generally, it is suitable to scan >10 times. The higher the number of scans, the higher the signal-to-noise ratio (S/N) and the more accurate the results, but those results in a longer time for spectral acquisition.

2) Air background is scanned to obtain a background spectrum.

3) The tissue under test is placed in tight contact with the ATR (the special pressure device is used). The testing is carried out.

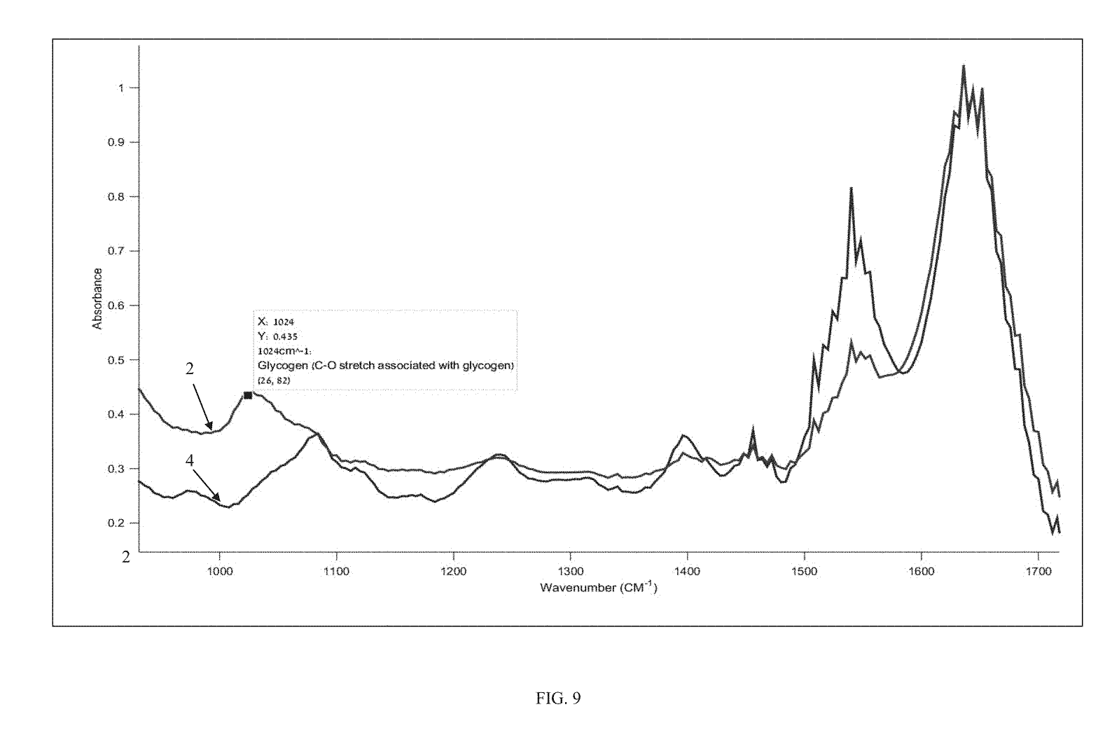

4) The spectrum that has been normalized by the background is obtained at the range 800-4000 cm⁻¹ of healthy persons and patients (see FIG. 9).

FIG. 9 graphically represents normal tissue 2 and cancerous tissue 4 in the “fingerprint” region 900-1800 cm⁻¹ with spectral biomarker increased in the glycogen content and changes in Amide1/Amide2 ratio.

The Measuring Procedure (Pre-Processing)

The tissue to be tested directly contacts the ATR element. The infrared light emitted from the IR source and passed through the ATR element forms infrared interference light after passing through the interferometer. After sampling, the infrared interference light with infrared spectral information enters into the detector part. The detector converts the infrared interference signals into electric signals which then are converted into digital signals by A/D conversion.

Finally, the spectrogram with infrared spectral information about the tissue is obtained by fast Fourier Transform and data processing.

The spectrogram is converted to the spectral absorbance, A(λ), as:

A(λ)=−log₁₀[R(λ)],

where

$$R ( λ ) = \frac{I ( λ ) - Dark ( λ )}{REF ( λ ) - Dark ( λ )} ,$$

REF(λ) is the reference signal (without sample);
Dark(λ) is the dark counts, and
I(λ) is the spectral intensity measured with the sample placed on ATR.

The detection of information is processed in connection with dedicated computer software, and it allows to instantly (during minutes) diagnoses whether the tested tissue has pathologic changes or not.

Methods for Tissue Diagnosis

Since the peak positions, peak widths, band shapes and relative intensities of spectra for tumor tissue may be different from healthy tissue, from a large number of spectrum data, the regularity of variations and the judgment criteria for diagnosis can be obtained.

The sophisticated statistical analyses can be used to assist in the identification of cellular types. For instance, several multivariate classification methods, including partial-least square (PLS), partial component regression (PCR), and linear discriminant analysis (LDA) have been shown to provide satisfactory results.

Among these multivariate methods, the LDA method may be preferred because it provides the best results and is simple to use.

In one embodiment, providing a diagnosis of the tissue includes forming an intensity spectrum. A diagnosis probability is computed based on intensities at particular wavelengths in the intensity spectrum. The diagnosis probability is compared to a threshold probability to characterize the tissue.

In a further embodiment, comparing the diagnosis probability to a threshold probability includes basing at least one of the diagnosis probability and the threshold probability on a logistics regression analysis, multivariate linear regression analysis (MVLR), stepwise regression analysis, best subset analysis, spectral peaks(s) ratio analysis, neural network analysis, or other analysis of data obtained from other tissue samples.

In a further embodiment, the pathological state of tissues can be considered by analysis of changes in band intensities, their ratios and places in different spectral regions:

a) Main molecular bonds:
amide-I (˜1650 cm⁻¹),
amide-II (˜1550 cm⁻¹),
amide-III (˜1240 cm⁻¹),
symmetric phosphate (˜1080 cm⁻¹),
glycogen (˜1030 cm⁻¹),
CH2 & CH3 of lipids (˜2852 cm⁻¹, ˜2923 cm⁻¹, ˜2960 cm⁻¹)

b) Main absorbance ratios as malignancy indicators:
glucose/phosphate (1030/1080),
glycogen/amide II (1045/1545),
Amide I/Amide II (1650/1550)
CH2/CH3 (2922/2960).

In a further embodiment, the diagnosis of the tissue is based on a database of tissue spectral absorbances.

According to further features in preferred embodiments of the invention, if it is not known apriori whether there is an abnormality, the method further includes the steps of detecting the abnormality according to a result of the data analysis.

The steps of detecting and analysis include Pre-processing and Classification based on Training.

In one embodiment, spectral data pre-processing may include subtraction of the water vapor spectrum, linear baseline corrections over well-defined regions (e.g., 2800-3100 cm−1 and 1800-900 cm−1), followed by a normalization of each spectral region to the maximum absorption band in that spectral region. A variety of spectral regions is examined separately or in combination to obtain optimal classification accuracy.

The developed software contains multiple analysis tools with the purpose of performing Grouping, Training and Classification.

The classification and training tools include:

a. Principal Component Analysis (PCA);

b. Support Vector Machine (SVM) for both training and classification; and

c. Discriminant Analysis Classification with the following kernels:
i. linear (LDA)—Fits a multivariate normal density to each group, with a pooled estimate of covariance;
ii. diaglinear—Similar to linear, but with a diagonal covariance matrix estimate (naive Bayes classifiers);
iii. quadratic (QDA)—Fits multivariate normal densities with covariance estimates stratified by group;
iv. diagquadratic—Similar to quadratic, but with a diagonal covariance matrix estimate (naive Bayes classifiers); and
v. mahalanobis—Uses Mahalanobis distances with stratified covariance estimates;

d. K-means clustering; and

e. PLSR—partial least squares regression.

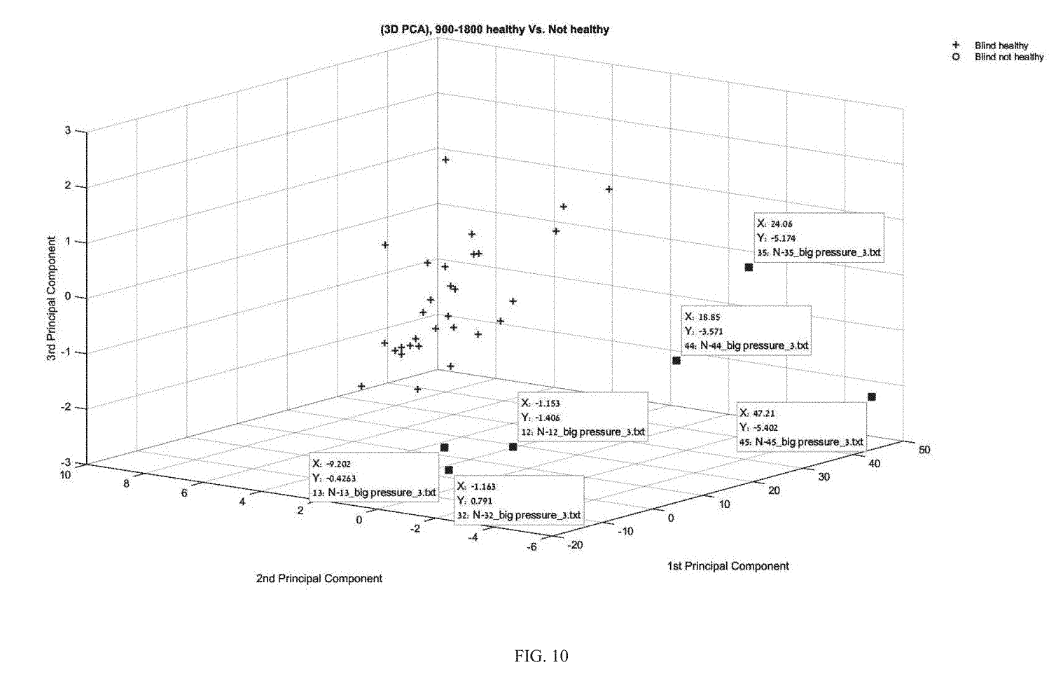

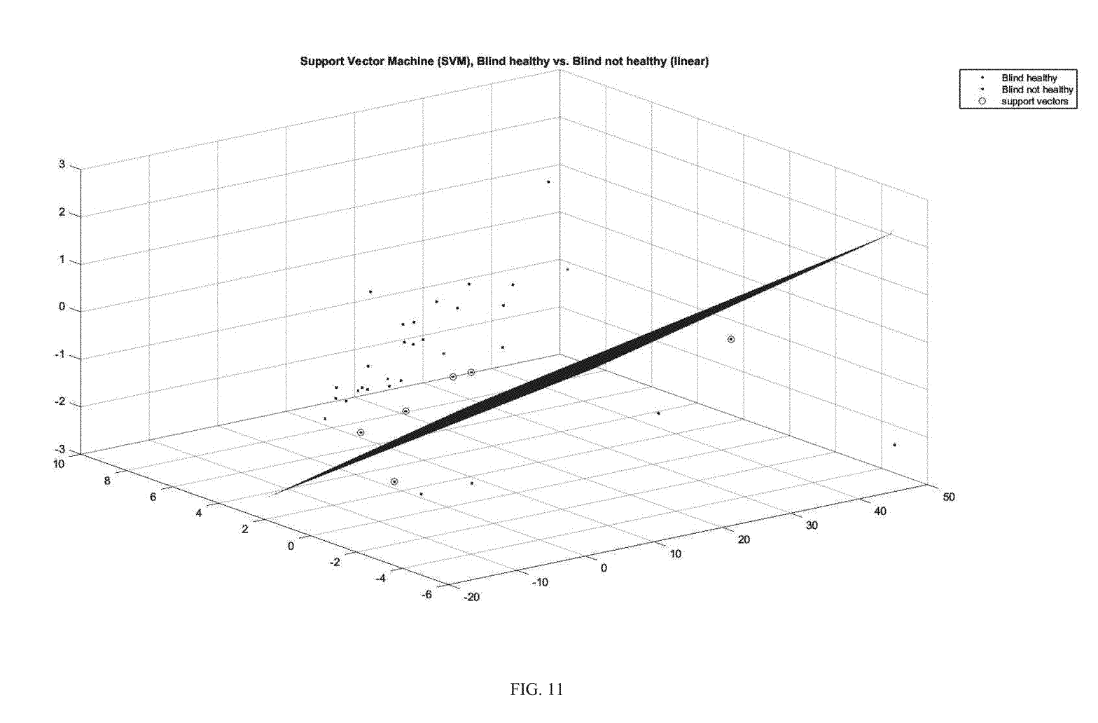

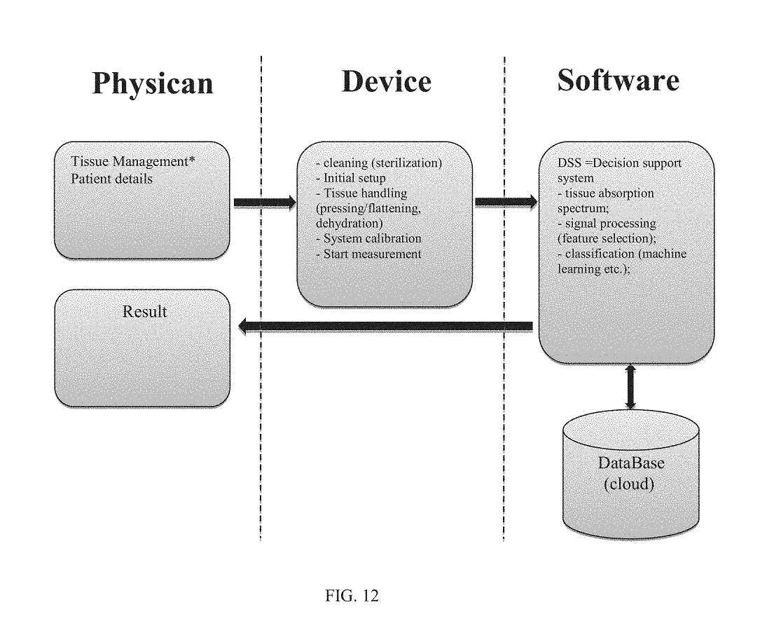

In a further embodiment, signals are grouped across different patients according to the clinical evaluation of the tissues; healthy and not healthy groups are generated. Samples are then analyzed using Principal Component Analysis (FIG. 10), a process which determines the basic spectral components of the signals. Support Vector Machine 2D/3D (FIG. 11) is used for both training and classification.

According to still further features in the described preferred embodiments, the step of processing includes calculating a differential measure.

According to still further features in the described preferred embodiments, the differential measure is a contrast. The magnitude of the contrast and its sign are used to assess normal cells and cells with pathological abnormalities. The contrast is investigated in specific wavebands to identify cancerous lesions and differentiate said cancerous lesions.

The contrast can be defined as a normalized difference of the spectral absorbance measured at two different wavelength bands Δλᵢ and Δλⱼ, where i≠j:

$$C = \frac{A ( {Δλ}_{i} ) - A ( {Δλ}_{j} )}{A ( {Δλ}_{i} ) + A ( {Δλ}_{j} )} ,$$

where A(λ) is the spectral absorbance at given wavelength.

In embodiments described above, the current invention may use the differential measure (contrast) between the normal cells and cells with pathological abnormalities as a classifier.

It should be noted that the device and method of the present invention do not require tissue management as required according to the current state of the art.

The steps of tissue management generally required presently include: (1) identification; (2) surgical resection; (3) getting the tissue sample into a properly labeled specimen container and (4) transportation to the pathology laboratory; (5) the specimen is accessioned into the pathology computer system and is (6) assigned a unique surgical pathology identifier. (7) The tissue is dissected by carefully cleaned instruments; (8) the specimen is divided among a number of tissue cassettes, small plastic containers, which are (9) labeled individually with the surgical pathology number and a unique block number.

However, the device and method of the present invention do not require steps 3-9 listed above.

Detection according to the innovative method is comprised solely of the following steps:

1) Set up operating parameters:
scanning rate;
the number of scans:
the resolution of the apparatus; and
spectral range.

2) Ambient background air is scanned to obtain a background spectrum.

3) The tissue under test is placed in tight contact with the ATR. A special pressure device is used as illustrated in FIG. 8. Calibration for maximal SNR is performed. The testing is carried out. Such pressure provides an optimal contact interface between the tissue sample and the face of the crystal. Applying pressure to the tissue sample also flattens the tissue so the IR EM wave can penetrate into all of the tissue and not miss any areas.

4) Drying—since water absorbs IR radiation, it is necessary to dry the tissue to get a better signal. Heat can harm the tissue and change the system readings because we are working in the thermal IR range. Therefore, we use a non-heat fan to dry the tissue as illustrated in FIG. 8.

5) System calibration—the mirrors are automatically adjusted to get the best SNR from the reflection angles (ATR).

6) The spectrum that has been normalized by the background is obtained at the range 800-4000 cm−1 (see FIG. 9).

It will be appreciated that the above descriptions are intended only to serve as examples and that many other embodiments are possible within the spirit and the scope of the present invention.

What is claimed is:

1. A device for real-time tissue diagnosis of biological tissue, the device comprising:
(a) a means for preparing a tissue sample before a measurement procedure that includes a tissue drying arrangement configured to at least reduce a moisture content of the tissue sample;
(b) a means for positioning an ATR element and mirrors so as to perform a system calibration;
(c) a means for irradiating a sample with IR radiation using said ATR element and an opto-mechanical assembly;
(d) a means for recording the absorption spectrum of a sample being tested;
(e) a means for carrying out a Fourier transformation of said absorption spectrum obtained into a FT-IR spectrum;
(f) a means for calculating tissue characteristics on the basis of signal processing;
(g) a means for comparing said characteristics from the step (f) in a pre-selected wavenumber range with the reference spectra prepared and stored in a database.

2. (canceled)

3. The device for real-time tissue diagnosis of biological tissue of claim 1, wherein said means for preparing a tissue sample before a measurement procedure includes a pressure device for ensuring good contact between said tissue sample and said ATR element.

4. The device for real-time tissue diagnosis of biological tissue of claim 1, wherein all mechanical components are deployed in a single portable benchtop housing.

5. A device for real-time tissue diagnosis of biological tissue, the device comprising at least an ATR element, mirrors, and a calibrating assembly with an automatic mirror position adjuster configured to adjust a position of at least one of said mirrors so as to perform a system calibration.

6. A method for real-time tissue diagnosis of biological tissue, the method solely comprising the following steps:
(a) setting operating parameters;
(b) scanning ambient background air to obtain a background spectrum;
(c) placing a tissue under test in tight contact with an ATR;
(d) drying said tissue so as to at least reduce moisture content of said tissue sample;
(e) automatically adjusting at least one system mirror thereby performing a system calibration; and
(f) obtaining a spectrum of said tissue sample.

7. The method for real-time tissue diagnosis of biological tissue of claim 6, wherein said setting operating parameters includes setting at least one of scanning rate; the number of scans; the resolution of the apparatus; and spectral range.

8. The method for real-time tissue diagnosis of biological tissue of claim 6, wherein said automatically adjusting at least one system mirror thereby performing a system calibration is a precise adjusting of a mirror position based on one of (a) a maximal signal for RAW data; (b) absorbance values; and (c) maximal SNR.
